(19) United States
(12) Patent Application Publication
(10) Pub. No.: US 2014/0341965 A1
(43) Pub. Date: Nov. 20, 2014

(54) Compositions and Methods Comprising Biodegradable Scaffolds and Retinal Pigment Epithelial Cells

(71) Applicant: Georgetown University, Washington, DC (US)

(72) Inventor: Nady Golestaneh, Bethesda, MD (US)

(73) Assignee: Georgetown University, Washington, DC (US)

(21) Appl. No.: 14/211,533

(22) Filed: Mar. 14, 2014

Related U.S. Application Data
(60) Provisional application No. 61/782,448, filed on Mar. 14, 2013.

(51) Int. Cl. A61L 31/16; A61K 35/30; C12N 5/079; A61L 31/04

(52) U.S. Cl. CPC A61L 31/16; A61L 31/044; A61K 35/30; A61L 31/047; C12N 5/0621; A61L 2300/64; C12N 2533/54; C12N 2535/10; C12N 2501/41; C12N 2501/415; C12N 2501/15; C12N 2501/40
USPC 424/426; 424/93.7; 435/398; 435/377

(57) ABSTRACT

The invention relates to compositions comprising a monolayer of functional retinal pigment epithelial (RPE) cells attached to a transplantable, biodegradable scaffold. The invention also relates to methods of using these compositions.

### BACKGROUND OF THE INVENTION

1. Field of the Invention

The invention relates to compositions comprising a monolayer of functional retinal pigment epithelial (RPE) cells attached to a transplantable, biodegradable scaffold.

2. Background of the Invention

Aged-related macular degeneration (AMD) is the leading cause of blindness among people over 55 years of age in the western world with tremendous social and economic consequences for the patients and their family. Caused by genetic and environmental factors, AMD progresses from the dysfunction and death of retinal pigment epithelium (RPE) cells to photoreceptor loss and deficits in sharp vision. Transplantation of functional RPE cells may hold promise in replacing the retinal pigment epithelium monolayer.

Studies have shown, however, that injection of RPE cell suspensions in animal models of retinal degeneration was not successful, as the transplanted cells died within 2 weeks and long-term survival was not achieved. Moreover, the monolayer structure that is essential to RPE function in vivo was never formed after transplantation. Therefore, injection of single cell suspension of RPE is not a promising method for cell-based therapy in AMD and there is a need for generation of transplantable and functional RPE monolayer.

What is needed is an implant comprising a biodegradable scaffold for use as a substrate for differentiation of cells into RPE cells that could, in turn, be directly transplantable into a subject in need of treatment without disturbing the RPE monolayer structure.

### SUMMARY OF THE INVENTION

The invention relates to compositions comprising a monolayer of functional retinal pigment epithelial (RPE) cells attached to a transplantable, biodegradable scaffold.

The invention also relates to methods of treating a subject suffering from macular degeneration, with the methods comprising transplanting into the subject a composition comprising a monolayer of functional retinal pigment epithelial (RPE) cells attached to a transplantable, biodegradable scaffold.

### BRIEF DESCRIPTION OF THE DRAWINGS

FIG. 1 depicts native RPE cells in culture on different type of nanofibers. Flat-single (A&D) and flat-double (B&E) fibers both supported RPE growth and survival, with the flat-double type (B&E) better facilitating monolayer formation (arrows). However, when flat-double fibers were coated with commercially available ECM proteins (matrigel) very few cells survived, probably because the fibers were masked by the ECM gelatinous consistence. Alternatively, the matrix proteins can be bioprinted on the fibers to obtain the most adequate matrix for RPE culture.

### DETAILED DESCRIPTION OF THE INVENTION

The invention relates to compositions comprising a monolayer of functional retinal pigment epithelial (RPE) cells attached to a transplantable, biodegradable scaffold.

As used herein, retinal pigment epithelial (RPE) cells are polar epithelial cells that exhibit both phenotypic and functional characteristics that are common and well-known to native RPE cells. The RPE cells produced by the methods of the invention need not exhibit every single characteristic of native RPE cells, but the characteristics of the RPE cells produced by the methods of the invention herein should be consistent with characteristics of native RPE cells. As used herein, “native RPE cells” are cells that have not been recombinantly manipulated in any way and naturally exhibit the phenotypic and functional characteristics of RPE cells. Native RPE cells can be found in in vivo and in vitro environments.

Phenotypic and functional characteristics of RPE cells include but are not limited to, presence or expression of melanin, presence or expression of pigment epithelium-derived factor (PEDF), presence of expression of RPE65, presence or expression of cellular retinaldehyde binding protein (CRALBP), presence or expression of bestrophin, presence or expression of Pax6 (although Pax6 is normally downregulated mature RPE cells), in the Na+/K+-ATPase being localized apically in the plasma membrane, the extracellular matrix metalloproteinase inducer (EMMPRIN) being located apically, N-CAM being located apically, αvβ5 integrin being located apically, chloride-bicarbonate exchange transporter being located basolaterally, Ca+-sensitive chloride channels being located basolaterally, syntaxin 2 (isoforms 2A and 2B) being located basolaterally, reduction or absence of syntaxin 3 expression, presence or expression of orthodentical homeobox 2 (OTX2), presence or expression of LIM homeobox 2 (LHX2), presence or expression of ectonucleoside triphosphate diphosphohydrolase 2 (ENTPD2), polarized secretion of vascular endothelial growth factor (VEGF), ability to form and maintain tight junctions, presence of a transepithelial potential (TEP), ability to perform phagocytosis, ability to form a confluent monolayer in culture, to name a few Other characteristics of RPE cells include, but are not limited to those characteristics discussed in Kokkinaki, M., et al., Stem Cells, 29:825-835 (2011).

The invention is not limited to the quality or quantity of characteristics of RPE cells for determining if the cells on the scaffold are functional RPE cells. The term “functional RPE cells,” means the cells at least have the ability to perform phagocytosis, express melanin and express RPE65. Of course, functional RPE cells can possess additional characteristics consistent with native RPE cells, such as those discussed in Kokkinaki, M., et al., Stem Cells, 29:825-835 (2011).

As used herein, the “presence or expression” of a particular protein or marker can be assessed by detecting or determining the protein levels. The “presence or expression” of a particular protein or marker can also be assessed by detecting or determining the by detecting or determining the mRNA levels that correspond to the gene or marker being expression. Alternatively, the “presence or expression” of a particular protein or marker can also be assessed with functional assays, whereby the cell is able to perform a specific function based on the presence of a functional protein or marker, for example, measuring a product or by-product from a chemical reaction in which the marker or protein being assayed takes part. Other functional assays that might be used to assess the presence or expression of a particular protein or marker might include measuring transmembrane standing potential at various places, e.g., apical or basal ends, in the cell. The invention is not limited by the methods of determining the presence or expression of a particular protein or marker. One of skill in the art can readily understand and appreciate numerous methods to determine the presence or expression (or absence thereof) of a particular marker or protein.

Methods of assessing functional characteristics of cells, for example to determine if cells are functional RPE cells, are well known in the art. For example, in vitro assays utilizing latex beads can be used to assess the ability of cells to perform phagocytosis. See Kilmanskaya, I., Meth. Enzymol., 418:169-194 (2006), which is incorporated by reference. Other in vitro assays include, but are not limited to, phagocytosis assays utilizing rod outer segments as described in Finnemann, S., et al., Proc. Nat'l. Acad. Sci., 94(24):12932-12937 (1997), which is incorporated by reference. In addition, polarity assays, for example to if the Na+/K+ATPase is located on the apical portion of the plasma membrane, are well known in the art and are discussed in Kokkinaki, M., et al., Stem Cells, 29:825-835 (2011), which is incorporated by reference.

In one embodiment, the cells that are initially seeded onto the scaffold are functional RPE cells or native RPE cells. In another embodiment, the cells that are initially seeded onto the scaffold are non-RPE cells.

As used herein “non-RPE cells” are cells that do not have all three characteristics of the ability to perform phagocytosis, expression of melanin and expression of RPE65. It is, however, possible that the non-RPE cells used in the methods of the present invention may exhibit one or more phenotypic or functional characteristics of native RPE cells. In one embodiment, the non-RPE cells used in the methods of the present invention do not express RPE65. In another embodiment, the non-RPE cells used in the methods of the present invention do not express melanin. In another embodiment, the non-RPE cells used in the methods of the present invention do not express melanin and do not express RPE65. In yet another embodiment, the non-RPE cells used in the methods of the present invention do not have the ability to perform phagocytosis, do not express melanin and do not express RPE65.

In one embodiment, the non-RPE cells used in the methods of the present invention are neither embryonic stem cells, nor are they induced pluripotent stem cells (iPSCs). In another embodiment, the non-RPE cells used in the methods of the present invention are embryonic stem cells induced pluripotent stem cells (iPSCs). In another embodiment, the non-RPE cells are not adult stem cells. In another embodiment, the non-RPE cells are mesenchymal stem cells. In another embodiment, the non-RPE cells are fibroblasts. The non-RPE fibroblasts used in the methods of the present invention can be derived from any connective tissue, including but not limited to, dermis, adipose, bone and cartilage. In one specific embodiment, the non-RPE fibroblasts cells are dermal fibroblasts. In another embodiment, the non-RPE cells are epithelial cells. The non-RPE epithelial cells used in the methods of the present invention can be derived from any epithelial tissue including, but not limited to, digestive system epithelium, skin epithelium, respiratory system epithelium, reproductive system epithelium and urinary system epithelium to name a few.

The source of the RPE or non-RPE cells can be any animal source; for example the source of the non-RPE cells can be human, non-human primate, canine, porcine, feline, bovine, equine rodent.

The scaffold supporting the RPE cells can be fabricated using, for example, polycapralactone (PCL) or other suitable biocompatible and biodegradable polymers such as, but not limited to, poly(L-lactic acid) (PLLA), poly (D, L-lactic-co-glycolic acid) (PLGA), poly (methyl methacrylate) (PMA), poly (glycerol sebacate) (PGS), poly (hydroxybutyrate-co-hydroxyvalerate), collagen, laminin, gelatin, and the like. The scaffold can also be fabricated with a blend of polymers. The polymer(s) is(are) formed into nanometer scale fibers, e.g., about 20-500 nm in diameter, but the fibers can be thicker. In one embodiment, the fibers are about 20 to about 50 nm in diameter. In another embodiment, the fibers are about 50 to about 100 nm in diameter. In another embodiment, the fibers are about 100 to about 200 nm in diameter. In another embodiment, the fibers are about 200 to about 300 nm in diameter. In another embodiment, the fibers are about 300 to about 400 nm in diameter. In another embodiment, the fibers are about 400 to about 500 nm in diameter. In another embodiment, the fibers are about 500 to about 700 nm in diameter. In another embodiment, the fibers are about 700 to about 1000 nm in diameter. In another embodiment, the fibers are about 1 to about 20 μm in diameter. In another embodiment, the fibers are about 20 to about 40 μm in diameter. In another embodiment, the fibers are about 40 to about 60 μm in diameter. In another embodiment, the fibers are about 60 to about 80 μm in diameter. In another embodiment, the fibers are about 80 to about 100 μm in diameter. In another embodiment, the fibers are 100 μm or more in diameter. In one embodiment, the scaffold is formed by extrusion of polymer(s) using an electrospinning process. Other methods of fabricating nanofibers are available and well known in the art and need not be repeated herein. To be clear, the invention is not limited by the methods of preparing the nanofibers for the scaffolding.

In one embodiment, the nanofibers of the scaffold are aligned nanofibers. The aligned fibers may be surrounded by a sheath of non-aligned, randomly oriented nanofibers. The RPE cells may be seeded in the sheath of non-aligned fibers, or on the aligned nanofibers. Methods of aligning nanofibers are well-known in the art and these methods can be used to produce scaffolds comprising aligned nanofibers. For example, Lee, P., et al., Biomed. Microdevices, 8(1):35-41 (2006), and U.S. Pre-Grant Publication No. 20100311949, which are incorporated by reference, disclose methods aligning collagen fibers for use in an in vitro cell culture setting. In addition, Wang, H. B., et al., J. Neural Eng., 6:016001 (2009) (available online at: iopscience.iop.org/1741-2552/6/1/016001/), which is incorporated by reference, discusses alignment of poly-L-lactic acid. Determining the extent of alignment of nanofibers is routine can be determined using well-known assays, such as but not limited to, Fast Fourier Transform (FFT) analysis.

In another embodiment, the nanofibers are spun onto a substrate. In one embodiment, the substrate is a hydrophilic substrate. In another embodiment, the substrate is a hydrophobic substrate. In yet another embodiment, the substrate is an amphipathic substrate. The substrate may or may not be composed of the same material composing the nanofibers. For example, PGS nanofibers may be spun onto a PGS substrate. Imprinting using inkjet technology is well known in the art. See, e.g., E. D. Ker et al., Biomaterials, 32, 3413 (2011), G. M. Cooper et al., Tissue engineering. Part A 16, 1749 (2010), J. A. Phillippi et al., Stem Cells 26, 127 (2008), E. D. Ker et al., Biomaterials 32, 8097 (2011), and E. D. Miller et al., Biomaterials 27, 2213 (2006), all of which are incorporated by reference.

In another embodiment, the nanofibers may be patterned onto a substrate. For example, the aligned fibers may be patterned as a triangular grid, square or rectangular grid, a pentagonal grid, a hexagonal grid, a heptagonal grid, an octagonal grid, a nonagonal grid and the like.

RPEs are known to secrete bioactive factors, such as but not limited to VEGF. These secreted factors may enhance growth rate and/or survival of RPE cells. As used herein, a bioactive factor is an agent or chemical entity that produces a biological effect on a cell or cells. The biological effect can be virtually any effect, such as, but not limited to, cell growth, apoptosis, induction of gene transcription, protein production, inhibition of protein production, cytokinesis. In one embodiment, the RPE cells can be the only source of bioactive factors. In another embodiment, the scaffold can be imprinted with one or more agents or bioactive factors, including but not limited to pigment epithelium-derived factor (PEDF), transforming growth factor-beta 1 (TGFβ1), dickkopf-related protein-1 or sonic hedgehog. The scaffolds can be imprinted with any combination of the factors listed herein.

In another embodiment, the scaffold can be imprinted with extracellualr matrix molecules (ECMs) such as not but not limited to fibronectin, vitronectin, matrix metalloproteinease-2, collagen I, collagen II, collagen III, collagen IV (including all substypes such as but not limited to IVA1, IVA2, IVA3, IVA4, IVA5, etc), collagen V, collagen VI, XVIII, elastin, heparan sulphate proteoglycan, laminin, integrins, Tissue Inhibitor of Metalloproteinase (I, II, Ill, IV, V) and the like. The scaffold can be imprinted with more than one ECM molecule.

In one embodiment, each side of the nanofiber scaffold is imprinted with the same ECM or bioactive factor(s). In another embodiment, each side of the nonfiber scaffold is imprinted with different ECM or bioactive factor(s). For example each side of the biodegradable scaffold can be biopatterned with specific ECM or bioactive factor(s) using well-established, custom inkjet-based biopatterning technology. Briefly, bioinks for the selected molecules are prepared using protocols previously described. In one embodiment, deposited matrix molecule concentrations can be modulated using an over-printing strategy as described previously in the art, whereby each location on the scaffold will be overprinted with a dilute bioink so that the deposited concentrations will increase proportionate to the number of drop over-prints. The inks absorb into and bind to the scaffold via native binding affinities. The fiber matrix of the scaffold is supported on an open PGS substrate frame, which will be held in place by a fixture that can be rotated to expose each side of the scaffold to the inkjets. Biopatterned constructs are storable dry, therefore the matrices can be shipped from one location to another where they can be biopatterned with matrix molecules.

The invention also relates to methods of culturing native RPE cells or induced RPE cells, comprising culturing the native or induced RPE cells on the scaffolds described herein. The scaffolds used in the cell culture methods described herein may or may not comprise aligned nanofibers.

The invention also relates to methods of treating a subject suffering from macular degeneration, with the methods comprising transplanting into the subject a composition comprising a monolayer of functional retinal pigment epithelial (RPE) cells attached to a transplantable, biodegradable scaffold. In particular, the methods comprise transplanting a composition comprising a biodegradable, transplantable scaffold and RPE cells to a subject in need of treatment of macular degeneration. In one embodiment transplantation into the subretinal spaces is accomplished using a posterior transscleral approach.

In another embodiment, the invention relates to treating Bruch's membrane (BM) to a subject in need thereof. These methods comprise generating a implanting a biodegradable scaffold into the subject, with the biodegradable scaffold comprising RPE cells. The biodegradable scaffold is a biopatterned scaffold made according to the methods of the present invention. For example, one side of the biopatterned scaffold comprises ECM or bioactive factors that support RPE growth, and the other side comprises ECM or bioactive factors that support RPE attachment and BM repair. In one specific embodiment, the side of the scaffold on which the RPE cells are the attach comprises at least fibtronectin that has been bioapatterned onto PGS scaffold. The scaffold comprises a PGS substrate and a PGS nanofiber matrix overlayed on top of the substrate.

### EXAMPLES

### Example 1

Sub-micron, oriented polystyrene STEP fibers are fabricated using the STEP pseudo-dry spinning process as is well known in the art. This technology enables precise tuning of fiber diameter in the range of 50-1000 nm by adjustment of processing parameters (rotational substrate velocity) and material parameters (polymer solution concentration, polymer molecular weight). Fiber spacing can be also controlled through adjustment of the translational substrate velocity. Fibers of up to centimeters in length can be easily generated. Highly aligned single (1D)- and double-layer (2D, crosshatch pattern) polystyrene fiber meshes are constructed.

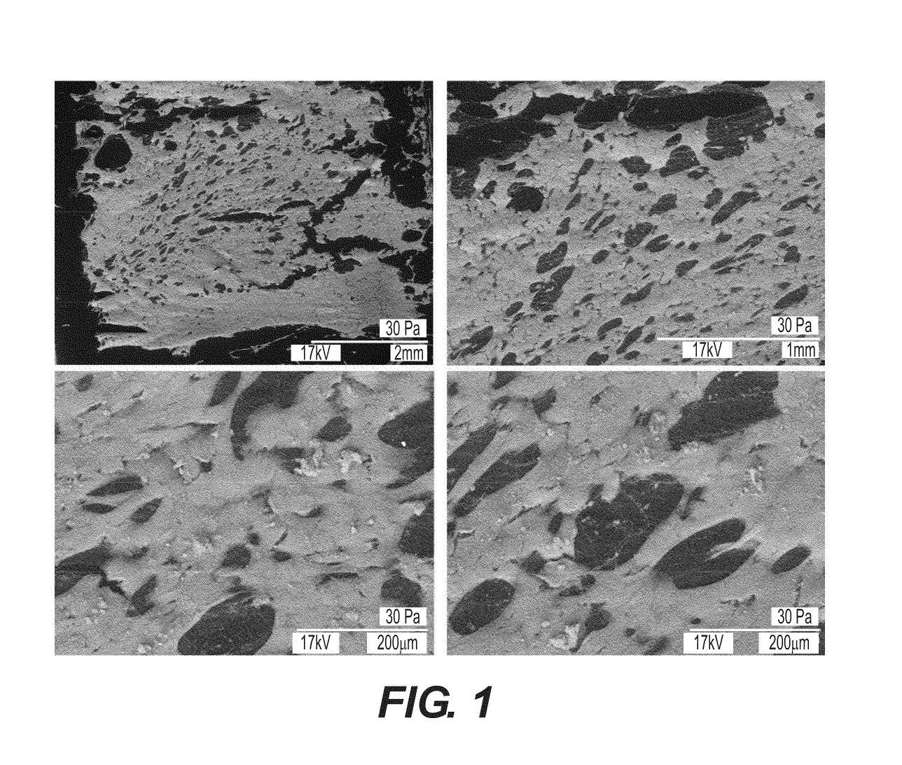

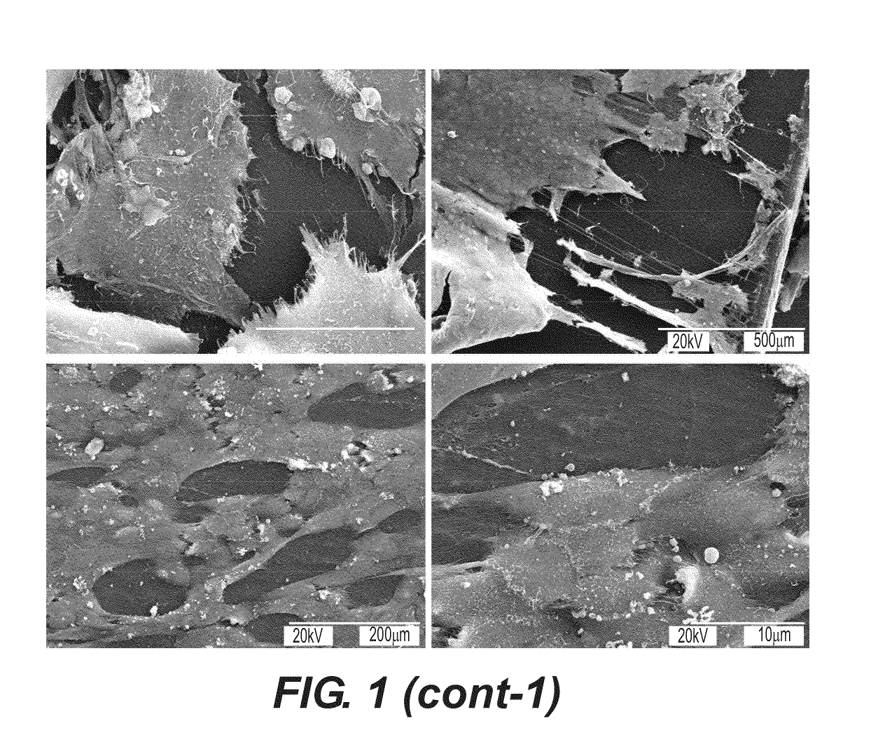

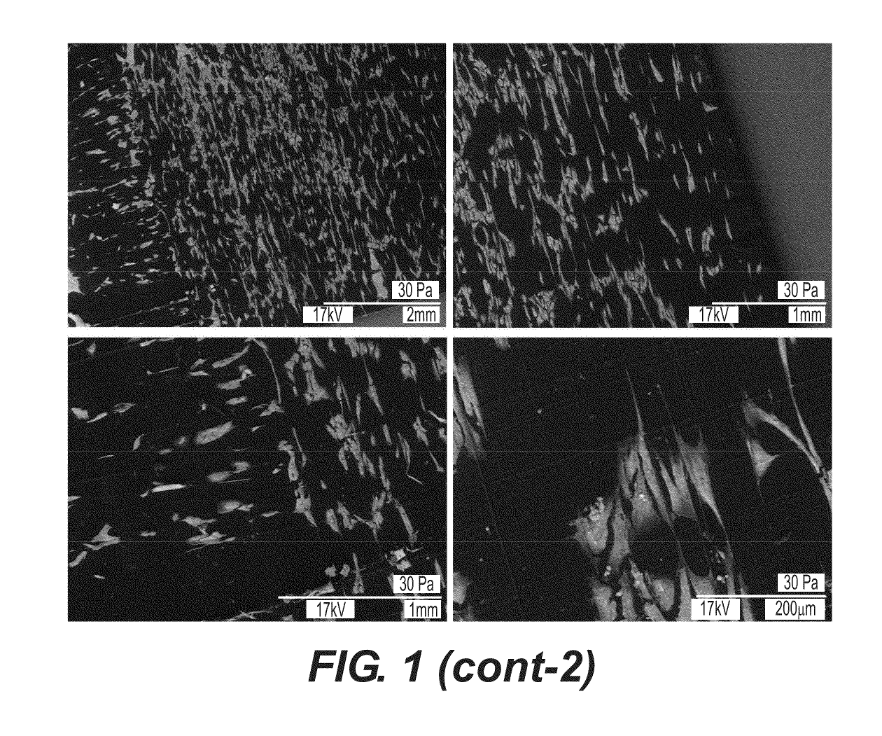

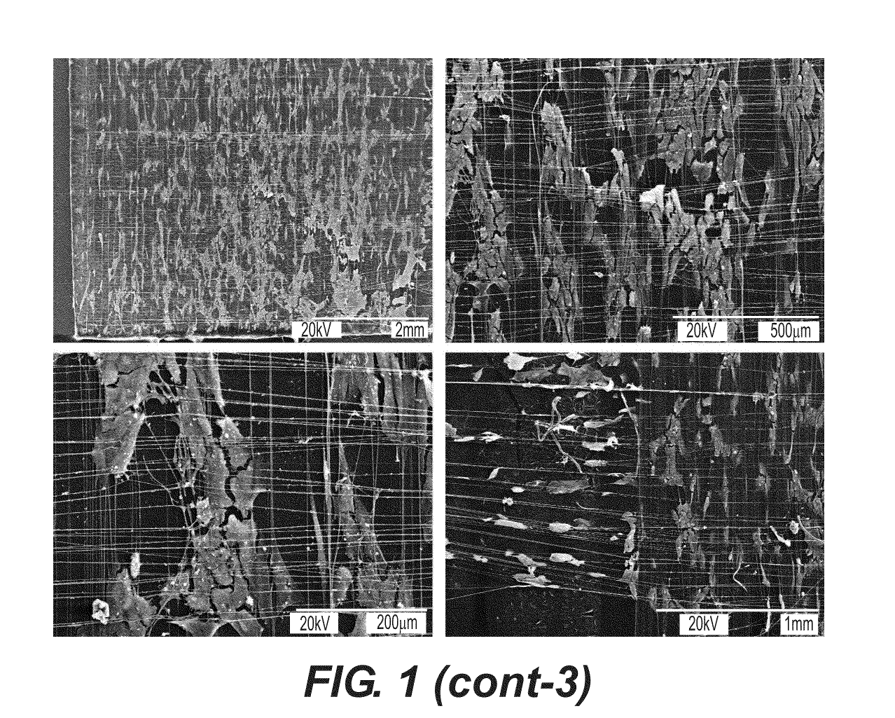

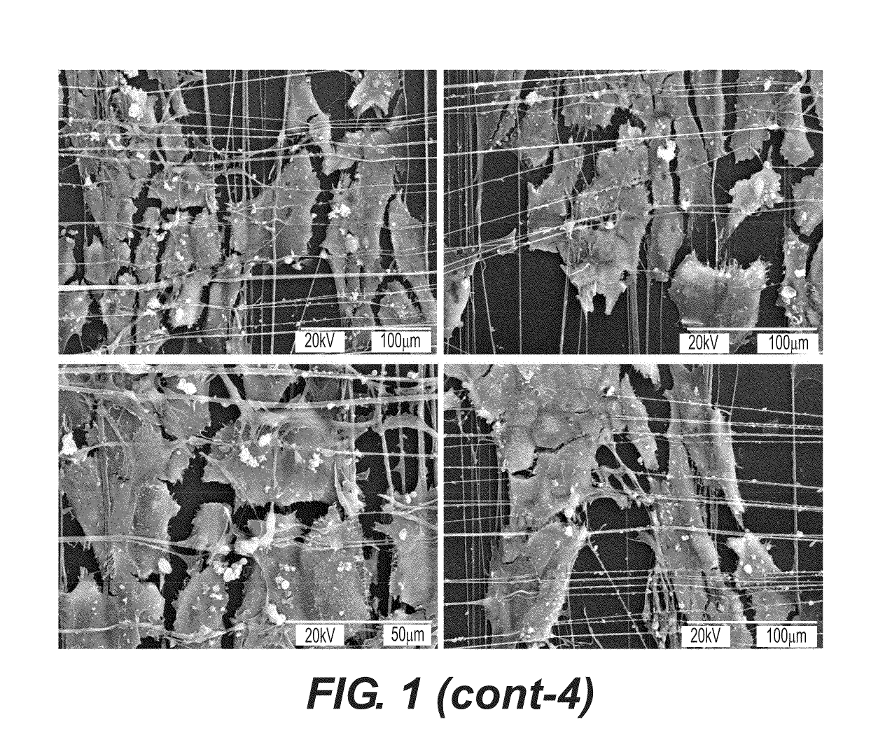

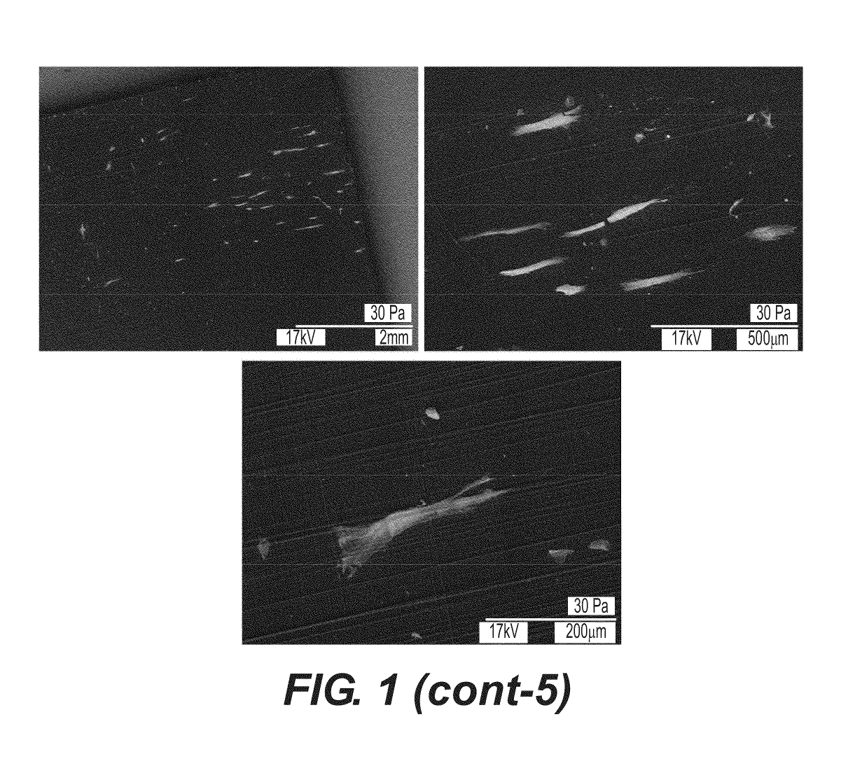

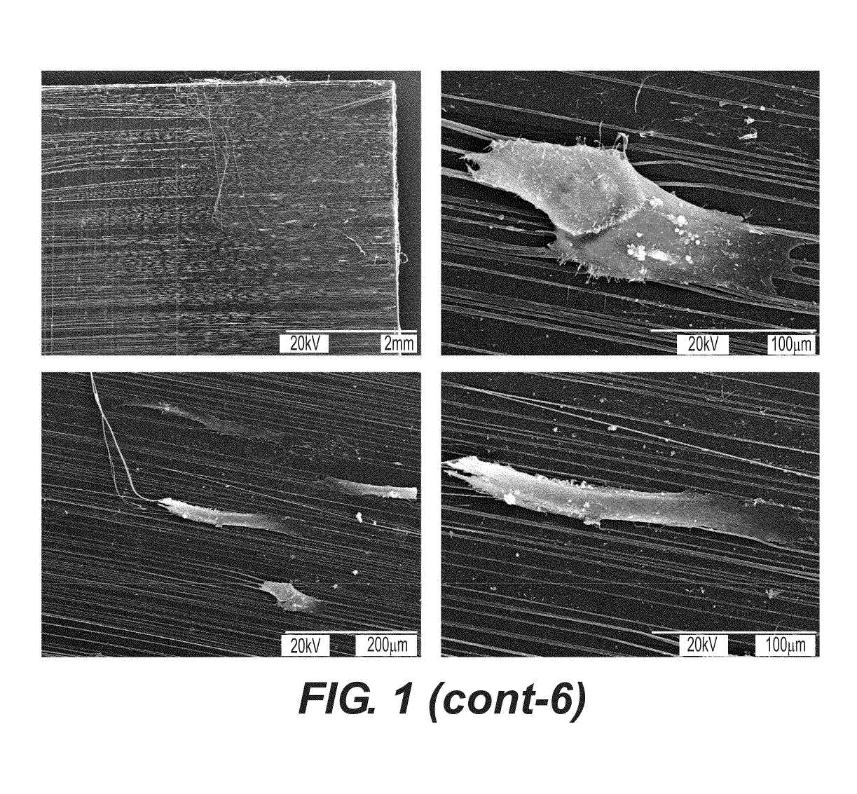

RPEs have been cultured on several types of fibers to determine the optimal geometry that promotes growth of the RPE monolayer. Four weeks after the seeding of RPE on the fibers, their morphology and monolayer structure was observed by scanning microscopy. As shown in FIG. 1, the “flatdouble layer” fibers are appropriate for RPE culture.

Next, fibers are imprinted with bioactive molecules using a combination of molecules including extracellular matrix proteins such as fibronectin and collagen IV. The optimum biochemical cues that support RPE monolayer growth and function are then selected. Prior to imprinting, matrix proteins are freshly diluted to the desired concentration in 10 mM sodium phosphate, pH 7.4. Prior to filling the inkjet with a specific matrix protein, the printhead is sterilized by rinsing with 70% ethanol followed by sterile deionized water. The bio-ink, consisting of 50-100 mg/ml MP is loaded into the printhead, and printed as is known in the art. Developing monolayers are screened by SEM and for pigment development as the primary selective criteria.

Using the information obtained above, fibers are generated that have correct geometry and biochemical cues and are made using biodegradable materials.

The in vivo viability and functionality of induced RPE cells (iRPEs) cultured on biodegradable matrices is tested by transplanting these monolayer cells into the subretinal zone of accepted mouse models of retinal degeneration. Retinal degeneration is induced by systemic injection of NaIO(3) to ablate the retinal pigment epithelial (RPE) layer in C57BI6 mice and to initiate neural retinal degeneration. Immunohistochemistry and live imaging analysis is performed to verify that the transplanted RPE is repopulating the retina and is forming an RPE monolayer. In addition, electroretinography (ERG) is used on the mice 2 months after transplantation to confirm improvements in vision function. Survival and function of photoreceptors following transplantation is also assessed.

### Example 2

The RCS rat is currently the most acceptable animal model for eye diseases with RPE dysfunction. It displays a phenotype of photoreceptor outer segment debris accumulation in the subretinal space, due to the inability of the retinal pigment epithelium (RPE) to phagocytose and shed photoreceptor outer segments. The phenotype is caused by a mutation in the receptor tyrosine kinase Mertk gene, which leads to a progressive loss of rod and cone photoreceptors. Photoreceptor outer segment shedding normally starts at postnatal day (P) 12 and a subretinal debris layer is apparent in the eyes of RCS animals at P20 (103-105). Thus the RCS rat animal model is the appropriate in vivo system that will allow us to investigate whether the iPSC-RPE cultured on a biodegradable and biopatterned matrix can integrate in the retina, form the appropriate cell junctions, attach to the BM and phagocytose the photoreceptor outer segments for vision to be improved or restored.

For transplantation, RCS rats are used at the age of 3-4 weeks when the dystrophic phenotype is apparent. Transplantation surgical is performed as is well known and previously described in the literature. Prior to surgery, the PGS scaffolds are rolled with adherent iPSC-RPE cells (2.0×2.0 mm) into a capillary glass tube of 2.0 mm diameter for injection via a microvolume syringe fit with a steel plunger, 6 cm in length and 2.0 mm in diameter. The rats are anesthetized with both general anesthesia and local anesthesia according to the established protocols. An incision (3.0-5.0 mm) is made in the lateral posterior sclera using a fine scalpel (5 mm blade). Through this sclerotomy the scaffolds are injected and unscrolled, taking care to position them into the subretinal space without disrupting the host retina. A single eye from each rat will receive the subretinal transplant. Finally, the scleral incision is closed with a suture, additional topical anesthetic is applied, and the rats are allowed to recover.

Electroretinography (ERG) is performed at various time points after transplantation (1, 2, 4, 6 and 8 weeks) to detect a delay in the deterioration of vision of the dystrophic RCS rats or a significant improvement in their eyesight. ERG is a well-established technique and has been described in detail for RCS rats in the literature. An Espion Visual Electrophysiology System ERG apparatus (Diagnosys, LLC) is sued to perform the ERG tests. The non-grafted eye will serve as a control for each rat.

The transplantation studies that have been published to date using cells on scaffolds or simple cell suspensions for retinal tissue engineering, have evaluated the viability of the transplanted cells by classical immunohistochemistry methods that preclude the possibility of monitoring the same eye in the live animal over a longer period of time post-transplantation. To overcome this limitation, the iPSC-RPE cells are labeled with stably integrated differentiation reporters (i.e., MITF promoter-GFP and RPE65 promoter—RFP) and live imaging of the RCS rats at 1-8 weeks after transplantation is performed. Live imaging experiments is performed using the Maestro II fluorescent imaging apparatus (CRI), equipped for isoflurane anesthesia and with a heated chamber and stage to maintain a stable body temperature.

At 4-8 weeks after surgery, animals will be euthanatized and the engrafted and control eyes will be processed for histology and immunohistochemistry based on established methods. The integration of the transplanted iPSC-RPE to the host retina is assessed by detecting GFP and RFP fluorescent markers on cryosections of the rat eyes. In addition, expression of RPE proteins (e.g. BEST1, RLBP1, OTX2) is detected by immunostaining with specific human antibodies to confirm that the transplanted cells retain their molecular phenotype in the host retina. To verify the integration of the transplanted cells in the host retina the photoreceptor outer segments are stained with anti-Rhodopsin and the capability of the transplanted iPSC-RPE for phagocytosis is evaluated by the percentage of iPSC-RPE that have endocytosed outer segments. To analyze the attachment of iPSC-RPE to the host BM, the BM is stained with anti-human integrin antibodies to detect the integrins, which would have been biopatterned onto the scaffold, and the rat anti collagen IV to verify the adhesion of biopatterned scaffold to the basal lamina of the host BM. In addition, since RPE express integrins and the iPSC-RPE are stably transfected to express RPE65-RFP, by immunostaining the BM with rat collagen IV, the interaction of iPSC-RPE integrins with collagen IV of the host BM is analyzed. The morphology of the host retina is also evaluated in sections of eyes that are embedded in paraffin or plastic by hematoxylin and eosin staining to determine if the scaffolds have introduced any morphological abnormalities to the host retina.

What is claimed is:

1. A composition comprising a monolayer of functional retinal pigment epithelial (RPE) cells attached to a transplantable, biodegradable scaffold.

2. The composition of claim 1, wherein the cells are induced RPE cells.

3. The composition of claim 1, wherein the transplantable, biodegradable scaffold comprises at least one material of collagen, fibronectin or laminin.

4. The composition of claim 1, wherein the transplantable, biodegradable scaffold is imprinted with at least one of PEDF, TGFβ1, dickkopf-related protein-1 or sonic hedgehog.

5. The composition of claim 1, wherein the transplantable biodegradable scaffold is comprised of nanofibers.

6. The composition of claim 1, wherein the transplantable biodegradable scaffold is comprised of aligned nanofibers.

7. A method of treating a subject suffering from macular degeneration or a deterioration of Bruch's membrane, comprising transplanting the composition of claim 1 into the subject.

8. A method of culturing native retinal pigment epithelial (RPE) cells or induced RPE cells, the method comprising seeding the native RPE cells or induced RPE cells on a biodegradable, transplantable scaffold.

9. The method of claim 8, wherein the transplantable, biodegradable scaffold comprises at least one material of collagen, fibronectin or laminin.

10. The method of claim 8, wherein the transplantable, biodegradable scaffold is imprinted with at least one of PEDF, TGFβ1, dickkopf-related protein-1 or sonic hedgehog.

11. The method of claim 8, wherein the transplantable biodegradable scaffold is comprised of nanofibers.

12. The method of claim 8, wherein the transplantable biodegradable scaffold is comprised of aligned nanofibers.

13. The method of claim 8, wherein the scaffold comprises a substrate and a nanofiber matrix, wherein the scaffold is imprinted with different extracellular matrix molecules or bioactive factors on each side of the scaffold.
